(19) United States
(12) Patent Application Publication
(10) Pub. No.: US 2023/0149443 A1
(43) Pub. Date: May 18, 2023

(54) CUSTOMIZABLE DOSAGE FORMS CONTAINING SIMETHICONE

(71) Applicant: Johnson & Johnson Consumer Inc., Skillman, NJ (US)

(72) Inventors: Peyton Hopson, Jacksonville, FL (US); Matthew A. Howard, Zionsville, PA (US)

(21) Appl. No.: 18/052,354

(22) Filed: Nov. 3, 2022

Related U.S. Application Data
(60) Provisional application No. 63/264,122, filed on Nov. 16, 2021.

(51) Int. Cl. A61K 31/80; A61K 9/20; A61K 31/451

(52) U.S. Cl. CPC A61K 31/80; A61K 9/2009; A61K 9/2072; A61K 9/2095; A61K 31/451

(57) ABSTRACT

An improved customizable dosage form comprising a substrate, such as a tablet core, that has one or more distinct, discreet cavities on its exterior surface, wherein simethicone is deposited into at least one of the cavities.

### CROSS-REFERENCE TO RELATED APPLICATIONS

This application claims the benefit of U.S. provisional application 63/264122 filed on Nov. 16, 2021, the complete disclosure of which is hereby incorporated herein by reference for all purposes.

### FIELD OF THE INVENTION

The present invention relates to a customizable dosage form containing ingredients with low melting temperatures and/or high viscosity and a process for making such customizable dosage forms wherein one or more active ingredients, colors, flavors and/or sensates are deposited into a cavity or cavities on the exterior surface of the dosage form.

### BACKGROUND OF THE INVENTION

There is a need in pharmaceutical industry processes to provide dosage forms which can combine high dose active ingredients with low dose active ingredients utilizing various portions of a tablet. Previous processes have often been cumbersome and costly, since they involve preparation of powders in separate unit processes, such as independent granulation steps which are later blended. Previous processes have also been limited to addition of an active ingredient to one portion of a tablet such in an additional compressed portion, in multilayer tablets, or in an outer coating. This can limit the amount or variety of active ingredients which can be combined.

There also is a need in the pharmaceutical industry to more easily combine ingredients, both active and inactive, into a single dosage form. Powder blends often contain incompatible ingredients in which multiple actives or active and inactive ingredients are in contact within the dosage form. This can lead to undesirable degradation of the active ingredient, especially under accelerated stability conditions.

There is also a need in the pharmaceutical industry to more easily work with ingredients that have low melting temperatures and high viscosity, such as simethicone. Historically, in preparing solid simethicone dosage forms, difficulties have been encountered when attempting to incorporate substantial quantities of the liquid simethicone in the solid final blend for tableting. The difficulty has been to achieve sufficient flowability for processing and sufficient cohesion for compaction, particularly for direct compression tableting, so that the tablet will withstand the rigors of further processing, e.g., film coating, gelatin dipping, printing, packaging and the like. Likewise, difficulties have been encountered in assuring that the viscous liquid simethicone is uniformly distributed throughout the solid formulation and expeditiously dispersed upon administration.

### SUMMARY OF THE INVENTION

The present invention provides an improved dosage form and process to deposit active and/or inactive ingredients with low melting temperatures and/or high viscosity on to such dosage forms. Particularly, the present invention provides a customizable dosage form comprising a substrate, such as a tablet core, that has one or more distinct, discreet cavities on opposing sides of its exterior surface. The present invention provides a customizable dosage from comprising a substrate, such as a tablet core, that has one or more distinct, discreet cavities on one side and an alignment feature on the opposing side. The present invention may also include an identification feature in addition to the alignment feature. The present invention also provides a process for making such a customizable dosage form wherein one or more active ingredients and inactive ingredients such as colors, flavors and/or sensates are deposited into at least one of the cavities. The present invention provides similar benefits as blended or granulated active ingredients, while providing the ability to vary and separate active ingredients from each other and active ingredients from inactive ingredients.

The present invention allows for deposition of active or inactive ingredients in one or more cavities in multiple regions across a tablet. The present invention allows for the deposition of simethicone into at least one cavity on the surface of the tablet, and immobilization of the simethicone portion. Use of the process to deposit ingredients with the invention provides advantages, including but not limited to, permitting the addition of actives, colors, flavors, sensates and textures; separating incompatible active and inactive ingredients; allowing for customization of dosage forms; compressing a substrate core without simethicone in the core or simethicone containing granulation, separating simethicone from other active ingredients or core ingredients, providing a perception of speed; permitting taste masking; and providing for visual recognition to aid in product selection.

### DETAILED DESCRIPTION OF INVENTION

As used herein, the term “dosage form” applies to any solid composition designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. The dosage form may be an orally administered system for delivering a pharmaceutical active ingredient to the gastro-intestinal tract of a human. The dosage form may also be an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

### Simethicone Background and Process Advantages

Simethicone has been used to treat intestinal discomfort, pressure, fullness, and bloating. It is typically administered in a liquid or solid form either alone or in combination with antacids or anti-diarrheals, such as loperamide.

Simethicone can be administered orally as a liquid preparation or as solid form for example capsules, chewable, swallowable or orally disintegrating tablets. One advantage of tablets over liquids is the ease of portability.

When administered orally, simethicone is used as an adjunct in the symptomatic treatment of flatulence, functional gastric bloating, and postoperative gas pains. The clinical use of simethicone is based on its antifoam properties. Silicone antifoams spread on the surface of aqueous liquids, forming a film of low surface tension and thus causing the collapse of foam bubbles. Thus, for self medication in over-the-counter preparations, simethicone is used as an antiflatulent to relieve symptoms commonly referred to as gas, including upper GI bloating, pressure, fullness, or stuffed feeling. It is often combined with other gastrointestinal medications, such as antacids, antispasmodics or digestive enzymes and various simethicone formulations are previously disclosed.

Japanese Patent No. SHO 39[1961]-46451 to Kitsusho Yakuhin Kogyo KK discloses a method for preparing simethicone tablets by mixing and granulating simethicone with aluminium silicate, magnesium aluminum metasilicate, and magnesium silicate. In particular, the formulation disclosed by the above Japanese patent requires at most 25% simethicone and 75% or greater silicate, binder and dispersing agent. Binders were disclosed as being starch and lactose. Dispersing agent was disclosed as being carboxymethylcellulose. Further, the above Japanese patent discloses that when the amount of simethicone exceeds 25%, a portion of the simethicone can be carried away, therefore the tablet workability is not desirable.

JP 5097681 to Horii Yakuhin Kogyo KK discloses a preparation wherein simethicone is adsorbed to magnesium aluminate metasilicate and dextrin. Excipient was then added and the preparation was tableted. Following tableting a hydroxypropyl methylcellulose phthalate coating was added, followed by applying additional simethicone and gelatin. The amount of simethicone in the final tablet was about 15%.

U.S. Pat. No. 4,906,478 discloses a simethicone preparation including a powdered combinate of particulate calcium silicate and simethicone. U.S. Pat. No. 5,073,384 discloses simethicone preparations including combinates of water soluble agglomerated maltodextrin and simethicone. U.S. Pat. No. 5,458,886 discloses a free-flowing granular composition including titanium dioxide having specific particle size and surface area in combination with simethicone.

U.S. Pat. No. 6,103,260 describes the use of an admixture of simethicone and either one or both of granular anhydrous tribasic calcium phosphate or dibasic calcium, wherein the admixture in a uniform granular composition of not more than 1000 micron particle size, that is suitable for compression into a solid dosage form for oral administration. While the amount of simethicone in the final composition was disclosed as being 10% to 50%, the final tablet weight was in excess of 1000 mg.

What is needed, therefore, is a compressible composition containing simethicone for forming a solid dosage, wherein either larger quantities of simethicone can be incorporated therein or smaller solid dosage forms containing the same amount of simethicone can be achieved.

Examples of suitable polydimethylsiloxanes, which include, but are not limited to dimethicone and simethicone, are those disclosed in U.S. Pat. No. 4,906,478, U.S. Pat. No. 5,275,822, and U.S. Pat. No. 6,103,260, the contents of each is expressly incorporated herein by reference. As used herein, the term simethicone refers to the broader class of polydimethylsiloxanes, including simethicone and dimethicone.

As used herein, simethicone conforms to the United States Pharmacopoeia (USP XXII) definition, that is a mixture of fully methylated linear siloxane polymers containing repeating units of polydimethylsiloxane stabilized with trimethylsiloxy end-blocking units, and silicon dioxide. Also, as used herein, dimethicone can be substituted for simethcone. Simethicone contains about 90.5-99% of polydimethylsiloxane and about 4-7% silicon dioxide. The polydimethylsiloxanes present in simethicone are practically inert polymers having a molecular weight of 14,000-21,000. The mixture is a gray, translucent, viscous fluid that is insoluble in water.

Historically, as noted above, in preparing solid simethicone dosage forms, difficulties have been encountered when attempting to incorporate substantial quantities of the liquid simethicone in the solid final blend for tableting. The difficulty has been to achieve sufficient flowability for processing and sufficient cohesion for compaction, particularly for direct compression tableting, so that the tablet will withstand the rigors of further processing, e.g., film coating, gelatin dipping, printing, packaging and the like. Likewise, difficulties have been encountered in assuring that the viscous liquid simethicone is uniformly distributed throughout the solid formulation and expeditiously dispersed upon administration.

Typically, to incorporate simethicone into a solid formulation, it must first be adsorbed onto a suitable porous carrier or substrate. There have been several inventions related to this problem where substrate materials vary from polysaccharides to inorganic materials such as calcium phosphates or metalo-silicates. A limitation of the polysaccharide approach is that the limited loading capacity i.e. a stable concentration of simethicone adsorbed onto the porous substrate resides in the range of 20-25% which implies a simethicone/adsorbate dose of 500-625 mg for a 125 mg dose of simethicone. A drawback of the inorganic substrates is their insolubility and gritty mouthfeel. Simethicone is hydrophobic, so it can affect the dissolution of certain active ingredients where co-formulated. It is desirable to have a composition wherein simethicone can be added to a solid dosage form without the use of a simethicone adsorbed composition.

The inclusion of a viscous fluid such as simethicone into the tableting process may result in sensitivities associated with pill breakage during packaging/customer opening, increased friability for subsequent processes, and water update. Additionally, the tableting process can cause an increased thermal load on the APIs such that specific degradants are formed. There are specific concerns associated with thermal degradation of simethicone into D4, D5, and D6 silicone degradants. The degradation mechanism can be either thermal (i.e., exposure to elevated temperatures), acid catalyzed, or base catalyzed.

One key aspect of the material choice is associated with the balance of surface wetting of the oil-soluble material while limiting diffusion into the material. This will be a function of material chemical composition (e.g., inter- and intramolecular interactions, etc.) and viscosity.

Simethicone can be administered orally as a liquid preparation or as solid form for example capsules, chewable or swallowable tablets. One advantage of tablets over liquids is the ease of portability. An advantage of swallowable tablets over chewable tablets includes the ease of ingestion and lack of taste. Coated tablets are preferred for swallowable tablets.

While it is possible to follow current tableting or gel filling technologies for inclusion of the class of materials such as simethicone, the following limitations exist:
Limited customization for API content without significant reformulation or equipment changeover
Potential increased thermal degradant products
Reduced excipient choice
Reduced shelf-life/stability for multi-active dosages
Tablet robustness (e.g., friability, breakage, etc.).

The items listed above suggest there are inefficiencies, limited flexibility, and increased cost for low melting material inclusion in solid dosage forms. As such, an alternative method should be considered that addresses the limitations detailed.

The strategies listed below may be used to immobilize a low melting temperature and viscous material such as simethicone within/on a solid dosage form:
a) Encapsulation of oil-soluble Active Pharmaceutical Ingredient (API) with phase change coating material
   After depositing the low melting temperature and/or viscous API into a predetermined cavity, a phase change material could be placed over the dosed material to encapsulate the API. The solidification process for the encapsulation material can consist of the following mechanisms:
      Melting/recrystallization
      Solution evaporation
      Ion-induced gelation
b) Encapsulation of water-soluble API with a phase change coating material
c) Encapsulation via covalently crosslinked material
   Following the same mechanism as encapsulation with a phase change coating material, wherein the material is a photocurable formulation
   This method could serve as a modified release coating
   Photocurable materials include the following:
      Methacrylated polydimethylsiloxane-co-polycaprolactone (mPDMS-co-PCL)
      Methacrylated polydimethylsiloxane-co-polyethylene glycol (mPDMS-co-PEG)
      Methacrylated polydimethylsiloxane-co-polylactic acid
      Methacrylated polydimethylsiloxane-co-polyglycolic acid
      Methacrylated polydimethylsiloxane-co-polydioxanone
      (Combinations of the above copolymers)
      Methacrylated polycaprolactone
      Methacrylated polyethylene glycol
      Methacrylated polylactic acid
      Methacrylated polyglycolic acid
      Methacrylated polydioxanone
      (Combinations of the above copolymers)
      Poly(ethylene glycol) diacrylate
      Gelatin metacryloyl
      Glycidyl-methacrylated hyaluronic acid
      Norbornene functionalized hyaluronic acid
      Norbornene functionalized poly(ethylene glycol)
      Norbornene functionalized aliphatic polyesters (e.g., PLGA, PDO, PCL, etc.)
d) Immobilization through prefabricated swellable network
   Matching solubility parameters between network and API.

An additional active agent may be added to the dosage form. The additional active may be selected from bisacodyl, famotidine, prucalopride, diphenoxylate, loperamide, lactase, mesalamine, bismuth, and pharmaceutically acceptable salts, esters, isomers, and mixtures thereof. The additional active may also be selected from calcium carbonate, magnesium hydroxide, magnesium oxide, magnesium carbonate, aluminum hydroxide, sodium bicarbonate, dihydroxyaluminum sodium carbonate and mixtures thereof.

Viscous low melting APIs for use in the present invention for deposition into the cavity include simethicone; Vitamin E also known as alpha-tocopherol or tocopherol; and Vitamin A also known as retinol or beta carotine.

### Tablet, Core and Cavity Definition

As used herein the term “tablet” refers to a solid form prepared by compaction of powders on a tablet press, as well known in the pharmaceutical arts. Tablets can be made in a variety of shapes, including round, or elongated, such as flattened ovoid or cylindrical shapes.

The core (or substrate) may be any solid form. The core may be prepared by any suitable method, for example the core be a compressed dosage form, or may be molded. As used herein, “substrate” refers to a surface or underlying support, upon which another substance resides or acts, and “core” refers to a material that is at least partially in contact with a portion of another material or surrounded by another material. For the purposes of the present invention, the terms may be used interchangeably: i.e., the term “core” may also be used to refer to a “substrate.” Preferably, the core comprises a solid, for example, the core may be a compressed or molded tablet, hard or soft capsule, suppository, or a confectionery form such as a lozenge, nougat, caramel, fondant, or fat based composition.

The core may have one or more major faces. The core may be in a variety of different shapes. For example, the core may be in the shape of a truncated cone. In other examples, the core may be shaped as a polyhedron, such as a cube, pyramid, prism, or the like; or may have the geometry of a space figure with some non-flat faces, such as a cone, cylinder, or the like. Exemplary core shapes that may be employed include tablet shapes formed from compression tooling shapes described by “The Elizabeth Companies Tablet Design Training Manual” (Elizabeth Carbide Die Co., Inc., p.7 (McKeesport, Pa.) (incorporated herein by reference) as follows (the tablet shape corresponds inversely to the shape of the compression tooling):
Shallow Concave.
Standard Concave.
Deep Concave.
Extra Deep Concave.
Modified Ball Concave.
Standard Concave Bisect.
Standard Concave Double Bisect.
Standard Concave European Bisect.
Standard Concave Partial Bisect.
Double Radius.
Bevel & Concave.
Flat Plain.
Flat-Faced-Beveled Edge (F.F.B.E.).
F.F.B.E. Bisect.
F.F.B.E. Double Bisect.
Ellipse.
Oval.
Capsule.
Rectangle.
Pentagon.
Octagon.
Diamond.
Arrowhead.
Bullet.
Barrel.
Half Moon.
Shield.
Heart.
Almond.
Parallelogram.
Trapezoid.
Figure 8/Bar Bell.
Bow Tie.
Uneven Triangle.

The core may be pressed of a blend of suitable active ingredients and excipients which may be either their natural color, including white, or can be conventionally colored as desired to provide a core of any desired color. Preferably the core contains loperamide HCl, for example 0.5 to 2.0 mg of loperamide.

As used herein the term cavity refers to a recess in the surface or face of a substrate or core designed to receive a deposited portion. The deposited portion may or may not contain an active ingredient.

The core of the present invention may comprise one or more cavities on multiple faces of the tablet, including opposing surfaces of the tablet.

The core may have any number and size of cavities on one or both surfaces of the tablet. The core may have up to 12 cavities on the tablet, including from about 1 to about 6 cavities on one surface of the tablet and from about 1 to about 6 cavities on the second surface of the tablet, including 6 cavities on one surface of the tablet and 6 cavities on the second surface of the tablet, 5 cavities on one surface of the tablet and 5 cavities on the second surface of the tablet, 4 cavities on one surface of the tablet and 4 cavities on the second surface of the tablet, 3 cavities on one surface of the tablet and 3 cavities on the second surface of the tablet, 2 cavities on one surface of the tablet and 2 cavities on the second surface of the tablet, 1 cavity on one surface of the tablet and 1 cavity on the second surface of the tablet. The core may have cavities on only one surface of the tablet.

The cavities on the core may be positioned so that they are in the same orientation or shape of the tablet. If the core is an elongated tablet shape, the cavities may also be elongated.

The cavities on the core may be physically separated by a portion of the surface of the core, with a portion of the surface between each cavity at least 1 mm, or at least 2 mm.

The term “deposition” and “deposited portion” refers to the placement and/or dispensing of a flowable material and said portion into a cavity of a dosage form, from a repository of said flowable material.

The deposited portion(s) may comprise simethicone. The dose of simethicone in the deposited portion(s) may be present in a dose from about 20 mg to about 200 mg, or from about 20 mg to about 125 mg of simethicone. The total simethicone dose may be separated into multiple cavities, for instance if a total of 125 mg of simethicone is present in the dosage form, 2 cavities may contain 62.5 mg in each cavity, or 3 cavities may contain 41.7 mg in each cavity, or 4 cavities may contain 31.25 mg in each cavity.

Since simethicone is present as an oil or emulsion, it is deposited in a composition that can be solidified in place through an encapsulation, photocuring, cooling, ionic gelation, drying or gelling step. The simethicone may be present in a melted solution or suspension, using a melted sugar, melted polymer or melted polyol. Suitable polyols include maltitol, sorbitol, erythritol, mannitol or xylitol. The ratio of simethicone to the polyol may be from about 10:90 to about 50:50.

The simethicone may also be present in a gelling solution, which is gelled upon deposition and dried in a separate step. Gelation may be achieved through cooling, addition of an ionic material of combination thereof. If an aqueous solution is used, the simethicone is present as a suspension or emulsion wherein the gelling agent is dissolved in solution. The simethicone may also be placed into a solvent based solution with other materials which aid in the solidification of the simethicone deposition. These materials may include polymers or surfactants.

The simethicone may be deposited in as a first portion within a cavity as a melt, aqueous solution or suspension, or solvent solution, and a separate portion of material may be added on top of the first portion containing simethicone in order to immobilize the simethicone through encapsulation or seal the simethicone deposition. The second portion may be substantially free of simethicone. As used herein substantially free is defined as less than 0.1%. weight by weight. The second portion may be a polymer, sugar or sugar alcohol or lipid which prevents the simethicone from migrating (e.g. immobilization) within the dosage form. The second portion may also contain a sensate, flavor or sweetener. Suitable materials for this encapsulation step include polymers such as cellulosic derivatives or polymethacrylates; or sugar alcohols.

With the methods above the immobilized simethicone may be free of particulates, whereas no particles greater than 30 microns are present in the deposited immobilized portion comprising simethicone. Particulates may be detected using methods such as microscopy or light scattering.

If a first cavity comprises simethicone, a second cavity may comprise loperamide.

### Alignment Feature and Identification Feature

As used herein the term alignment feature refers to a recess or protrusion in the surface or face of a substrate or core that is designed to orient a tablet during the manufacturing process.

The alignment feature may be any shape that allows for consistent seating or orientation of the tablet throughout the manufacturing process. The alignment feature may be a triangle, rectangle, elongated diamond, trapezoid or star. The alignment feature may be placed at the center of the tablet surface. The alignment feature may be placed off-center on the tablet surface. The alignment feature may be placed at or near the edge of the tablet surface.

The alignment feature may be a recess or hollow portion of the tablet surface that is designed to receive or be seated in a corresponding shape. The alignment feature may be a protrusion or raised portion of the tablet surface that is designed to be inserted into a corresponding recess or hollow.

The alignment feature may allow for better precision, accuracy and speed during deposition of ingredients into the cavities.

As used herein the term identification feature refers to any markings, letters or numbers or combinations thereof that provide information to a consumer about the dosage form. Such information may include active ingredient, amount of active ingredient, manufacturer and/or brand name.

### Core Ingredients

The core may contain a disintegrant and/or a superdisintegrant. Suitable disintegrants for making the core, or a portion thereof, by compression, include, e.g., sodium starch glycolate, cross-linked polyvinylpyrrolidone, cross-linked carboxymethylcellulose, starches, microcrystalline cellulose, and the like. The superdisintegrant may be present as a percentage of the weight of the core from about 0.05 percent to about 10 percent.

The dosage form of the present invention preferably contains one or more active ingredients. Suitable active ingredients broadly include, for example, pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, anti-smoking agents, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, oncology agents, osteoporosis preparations, polydimethylsiloxanes, respiratory agents, sleep-aids, urinary tract agents and mixtures thereof.

Suitable flavorants include menthol, peppermint, mint flavors, fruit flavors, chocolate, vanilla, bubblegum flavors, coffee flavors, liqueur flavors and combinations and the like.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotidine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate, loperamide and racecadotril; glycopyrrolate; antiemetics, such as ondansetron, analgesics, such as mesalamine.

At least one active ingredient may be selected from bisacodyl, famotidine, ranitidine, cimetidine, prucalopride, diphenoxylate, loperamide, lactase, mesalamine, bismuth, antacids, and pharmaceutically acceptable salts, esters, isomers, and mixtures thereof.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors should be considered, as known in the art. Typically, the dosage form comprises at least about 1 weight percent, preferably, the dosage form comprises at least about 5 weight percent, e.g., about 20 weight percent of one or more active ingredients. The core may comprise a total of at least about 25 weight percent (based on the weight of the core) of one or more active ingredients.

The active ingredient or ingredients may be present in the dosage form in any form. For example, one or more active ingredients may be dispersed at the molecular level, e.g., melted or dissolved, within the dosage form, or may be in the form of particles, which in turn may be coated or uncoated. If an active ingredient is in the form of particles, the particles (whether coated or uncoated) typically have an average particle size of about 1-2000 microns. Such particles may be crystals having an average particle size of about 1-300 microns. The particles may also be granules or pellets having an average particle size of about 50-2000 microns, preferably about 50-1000 microns, most preferably about 100-800 microns.

The dissolution characteristics of the at least one active ingredient may follow an “immediate release profile”. As used herein, an immediate release profile is one in which the active ingredient dissolves without substantial delay or retardation due to the dosage form. This can be contrasted with the dissolution of modified release, e.g., delayed or controlled release dosage forms known in the art. The dissolution rate of the immediately released active ingredient from the dosage form of the invention may be within about 20% of the dissolution rate of the active ingredient from a pure crystalline powder of said active ingredient, e.g., the time for 50%, 75%, 80%, or 90% dissolution of active ingredient from the dosage form is not more than 20% longer than the corresponding time for 50%, 75%, 80%, or 90% dissolution of active ingredient from a pure crystalline powder of said active ingredient. The dissolution of the immediately released active ingredient from the dosage form may also meet USP specifications for immediate release tablets, gelcaps, or capsules containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing; and for acetaminophen and codeine phosphate capsules USP 24 specifies that at least 75% of the acetaminophen contained in the dosage form is dissolved within 30 minutes in 900 mL of 0.1 N Hydrochloric acid using USP Apparatus 2 (paddles) at 50 rpm; and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes. See USP 24, 2000 Version, 19 - 20 and 856 (1999). The immediately released active ingredient may be acetaminophen, and when tested in 37° C. water using USP Apparatus II (paddles) at 50 rpm, at least 80%, preferably at least 85%, of the acetaminophen contained in the dosage form is released therefrom within 30 minutes.

The time for release of at least 80%, preferably at least 85%, of at least one active ingredient contained in the dosage form is released therefrom may not be more than about 50%, e.g., not more than about 40% of the time specified by the dissolution method for immediate release listed in the United States New Drug Application for that particular active ingredient.

When the immediately released active ingredient is acetaminophen, when tested in 37° C. water using USP Apparatus II (paddles) at 50 rpm, at least 80% of the acetaminophen contained in the dosage form is released therefrom within about 6 minutes, e.g., within about 5 minutes, or within about 3 minutes.

The tablet and deposited portions can be observed using the USP Disintegration test as outlined in USP 34- NF29, Section 701. The tablet and coating positions can also be observed by placing the tablet into water at 37° C. without agitation.

Disintegration of the tablet without agitation can be observed at less than about 30 seconds, e.g., less than about 15 seconds, e.g., less than about 10 seconds, e.g., less than about 5 seconds.

The tablet of the present invention may be observed using a defoaming test, wherein the simethicone portion contributes to defoaming to aid in the minimization of intestinal gas. A suitable defoaming test includes one described for simethicone tablets in the United States Pharmacopeia (USP 29 NF24) incorporated herein by reference. Using this procedure, the tablets of the present invention do not exceed a defoaming time of 15 seconds.

The core may be covered with a coating that can be any number of medicinally acceptable coverings. The use of coatings is well known in the art and disclosed in, for example, U.S. Pat. No. 5,234,099, which is incorporated by reference herein. Any composition suitable for film-coating a tablet may be used as a coating according to the present invention. Examples of suitable coatings are disclosed in U.S. Pat. Nos. 4,683,256, 4,543,370, 4,643,894, 4,828,841, 4,725,441, 4,802,924, 5,630,871, and 6,274,162, which are all incorporated by reference herein. Suitable compositions for use as coatings include those manufactured by Colorcon, a division of Berwind Pharmaceutical Services, Inc., 415 Moyer Blvd., West Point, PA 19486 under the tradename “OPADRY®” (a dry concentrate comprising film forming polymer and optionally plasticizer, colorant, and other useful excipients). Additional suitable coatings include one or more of the following ingredients: cellulose ethers such as hydroxypropylmethylcellulose, hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as Polysorbate-80, sodium lauryl sulfate and dioctyl-sodium sulfosuccinate; polycarbohydrates, pigments, opacifiers.

Preferred coatings include water soluble polymers selected from the group consisting of hydroxypropylmethyl cellulose, hydroxypropyl cellulose, methyl cellulose, polymethacrylates, polyvinyl alcohol, polyvinyl alcohol:polyethylene glycol copolymers and mixtures thereof. These water soluble coating polymers are also suitable for the encapsulation method of the present invention, wherein the simethicone is deposited and sealed or encapsulated with a polymer.

The average thickness of the coating is preferably in the range from about 1 to about 150 microns, or from about 50 to about 90 microns, or from about 10 to about 90 microns, or from about 20 to about 80 microns, or from about 30 to about 70 microns.

The coating may comprise from about 10 percent to about 50 percent, e.g., from about 15 percent to about 20 percent of HPMC. The dried coating typically is present in an amount, based upon the dry weight of the core, from above about 0 percent to about 5 percent, or from about 1 percent to about 4 percent, or from about 2 percent to about 3 percent, or from about 1 to about 2 percent. The coating composition is optionally tinted or colored with colorants such as pigments, dyes and mixtures thereof.

A layer of coating may be applied to the entire exterior surface of core prior to application of the deposited portion. Coating can be applied as a clear, transparent coating such that the core can be seen. The choice is dictated by the preference of the manufacturer and the economics of the product. A commercially available pigment may be included the coating composition in sufficient amounts to provide an opaque film having a visibly distinguishable color relative to the core. The coating may be added after the deposited portion is added to the tablet.

### Deposited Portion

The tablet of the present invention may comprise from about one to about four cavities on one or both major faces. The cavities may comprise a deposited portion. The cavity may be designed to receive a deposit of up to about 50 mg (or 0.05 mL of solution), or up to about 10 mg (or 0.01 mL of solution).

In the present invention, an active ingredient is first incorporated into a flowable form or flowable material so that it can be deposited (as a deposited portion) in the cavity(ies) of the tablet. The flowable form may be a solution, emulsion, gel, suspension, melted solution, melted suspension, or semisolid. The flowable form is subsequently solidified via cooling, drying or a mixture of both to become a final deposited portion.

The deposited portion of the present invention may comprise at least one polymer. In addition, the deposited portion may comprise a surfactant. Suitable surfactants may include nonionic surfactants such as sorbitan esters, polysorbates, or poloxamers.

The tablet comprising deposited portions of the invention provides an observable means of differentiation. The term “observable” (and forms thereof such as “observably,” “observing,” etc.) is intended to have its common meaning, i.e., perceptible (or “perceptibly,” perceiving,” etc. as appropriate) using any one or more of the five human senses, e.g., sight, sound, touch, taste and smell. The tablet comprising deposited portions described herein can employ interaction with one or more of the five senses, and particularly may employ visual, audible and tactile interaction or combinations thereof. Preferably, the tablet comprising deposited portions employs interaction with the visual sense.

The deposited portions of the present invention may comprise at least one active ingredient. The deposited portion may comprise two or more active ingredients. The deposited portion may comprise an inactive ingredient such as a sweetener, a flavor, a color or sensate which is separate and distinct from the sweetener, flavor, color or sensate in another deposited portion on the surface of the tablet. The deposited portion may be substantially free of a pharmaceutical active ingredient and contain an inactive ingredient such as colorant and/or a sweetener, flavor, sensate or mixture thereof.

The deposited portions of the present invention may be measured for consistency and accuracy in a variety of ways. The deposited portion may be measured as a function of “spread”, wherein the deposited portion spreads to a percentage of the area within the cavity, which is then measured as that which is covered by the deposited portion. The percentage of area in which the deposited portion spreads within at least one cavity may be at least 50 percent, or at least about 60 percent or at least about 70 percent.

In some cases the cavity spread has the undesirable effect of being greater than 100 percent of the area of the cavity. This may occur with uncoated tablets. In some cases the cavity spread is between 70 percent and 100 percent with a tablet comprising at least 0,1 percent of a film coating by weight of the core.

Another measure of the deposited portion is that of diffusion, or the level or length at which the deposited portion diffuses into the body of the tablet. This can be measured by adding a colorant to the deposited portion solution, suspension or mixture which is different than the color of the tablet core. This can also be measured through other spectroscopic methods such as FTIR or Raman spectroscopy. The diffusion of the deposited portion may be less than 30 mm², or less than 20 mm², or less than 10 mm². In some cases the diffusion is greater when using a coated tablet versus an uncoated tablet, wherein the diffusion is more than 5 percent greater when dispensing on an uncoated tablet versus a tablet that comprises at least 0.1 percent of a film coating by weight of the core.

Another measure of the deposition is the amount of cross-sectional surface area of the tablet that is occupied by the deposited portion. The percentage of area of the tablet may be at least 5 percent, or at least 10 percent, or at least 20 percent of the surface.

Another measure of the deposited portion is that of tablet swelling. Tablet swelling is measured by the percentage of thickness which is increased by the addition of the deposited portion. In the current invention the level of swelling is less than 10 percent, or less than 5 percent.

Multiple deposition steps may be performed within each cavity to create the deposited portion(s). The deposited portion may be created through one to ten deposition steps, or between one and five deposition steps, or between one and three deposition steps.

The tablet comprising deposited portions of the invention can provide a mechanism by which consumers are provided with criteria that are relevant to appropriate selection or deselection of a given product. For example, the tablet comprising deposited portions is presented to the consumer and the consumer simply visually observes decision criteria and selects or deselects a product based on the criteria. Any type of design which functions as a cue as described herein is encompassed by the instant invention.

The “criteria” will have relevance to the decision-making process for deciding whether or not a product is appropriate for, and therefore could be purchased and used by, a consumer considering using the product. Since different criteria for use will apply to different products, the criteria will vary depending on the product being marketed. Examples of criteria include but are not limited to drug, location of symptoms, symptoms treated, time of day for use, drowsy/non-drowsy, form, flavor and combinations thereof.

Criteria as used herein includes both single (i.e., criterion) and multiple (i.e., criteria) characteristics on which a decision may be based. Therefore, criteria may include single or multiple characteristics which are relevant to the decision making process.

Each of the selectable responses will be either positively associated with appropriate purchase and use of the product by a consumer (i.e., a positive selectable response), or negatively associated with appropriate use (i.e., a negative selectable response) and therefore would be associated with deselection of the product.

The term “selection indicia” is intended to mean any observable symbol which is either positively associated with appropriate purchase and use of the product, i.e., positive selection indicia, or negatively associated with appropriate purchase and use of the product, i.e., negative selection indicia. Selection indicia include observable symbols such as graphic symbols including color coding, alphanumeric graphics, pictorial graphics and the like, and sounds such as musical notes, bells, audible language and the like, and combinations thereof. The selection indicia are chosen to be compatible with the design of the dosage form.

For the sake of brevity, the term “indicia” as used herein includes both single symbols (i.e., indicium), such as a single color or graphic, and combinations of symbols (i.e., indicia), such as stripes of alternating colors or a specific color background with a pictorial and/or alphanumeric graphic in the foreground, and the like. Therefore, a single selection indicia may be comprised of one symbol or a combination of symbols which, when observed together as a whole, serve as a single positive or negative selection indicia.

The dosage form of the present invention may be a multilayer tablet, e.g., a trilayer tablet or a bilayer tablet. A bilayer tablet may comprise a modified or sustained release layer and an immediate release layer.

The deposited portion may be comprised of a material that is melted and solidifies upon application of the deposited portion. The deposited portion may cool and harden at room temperature or upon cooling at a temperature less than 25° C. Suitable low-melting hydrophobic materials include polymers, thermoplastic carbohydrates, fats, fatty acid esters, phospholipids, and waxes. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GLYCOWAX-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include carnauba wax, spermaceti wax, beeswax, candelilla wax, shellac wax, microcrystalline wax, and paraffin wax; fat-containing mixtures such as chocolate; and the like.

Suitable meltable polymers include hydroxypropyl methylcellulose, hydroxypropyl cellulose, hydroxypropyl methylcellulose acetate succinate, cellulose acetate, ethyl cellulose, polyacrylic acid, polyvinyl alcohol, polyvinylpyrrolidone, pluronics, poloxamers, polyethylene oxide, polyvinyl acetate, polylactic acid and polycaprolactone, and copolymers thereof.

Some active ingredients may be only partially soluble in water and are better suited to deposition in a melt or solvent based deposition system. Suitable active ingredients include but are not limited to doxylamine and chlorpheniramine maleate.

The deposited portion may contain a carbohydrate which melts and flows below 200° C., preferably below 150° C., or below 120° C., e.g., “meltable”. Suitable meltable carbohydrates include polysaccharides such as polyfructose, polydextrose, inulin, hydrogen starch hydrosylate; isomalt or sugar alcohols such as xylitol, sorbitol, maltitol, erythritol and mixtures thereof.

The deposited portion may be applied as a solvent based solution, and the solvent is subsequently dried off after application to the dosage form. The solvent may comprise ethanol, methanol, hexane, cyclohexane, isopropyl alcohol, dichloromethane, acetonitrile, tetrahydrofuran or acetone. The solution may comprise a hydro-alcoholic system, combining alcohol with water. The solution can also comprise the polymer, carbohydrate, plasticizer, wax, active ingredient and mixtures thereof.

The deposited portion may comprise a gelling material, or a material that solidifies into a gel upon deposition. The deposited portion may comprise a crosslinked hydrogel material. The dosage form is exposed to visible and/or ultraviolet light after deposition of the appropriate photocurable formulation. The solution can comprise of a photoinitiator, solvent, inhibitors, photocurable oligomer or monomer, light absorber and mixtures thereof. The dosage form is then dried after deposition to remove the water, solvent or combination of both. Suitable gelling materials may include gelatin, pectin, gellan gum, carrageenan, guar gum and xanthan gum. Suitable water soluble film forming polymers for use in the dosage form include but are not limited to poloxamers, polyvinyl alcohol, hydroxypropyl cellulose, hypromellose, methylcellulose, pullulan, modified starches, and hydroxyethylcellulose.

In the case of ionic gelation step, the gelling materials described above would be suitable. In the case of the ionic gelation method the gelling material may be solidified or cross-linked using an ionic material such as a salt. Suitable salts would include ingestible sodium, calcium, magnesium or potassium salts.

The deposited portion may disintegrate at a different rate than other deposited portions or the core. Where the deposited portion is immediate release, the portion may disintegrate in less than 60 seconds, or less than 30 seconds, or less than 10 seconds. Disintegration testing may be performed using the apparatus and method described in General Chapter 701 of the United States Pharmacopoeia, more specifically the edition USP 43-NF 38.

### Process

One preferred process of manufacturing intermediate dosage form begins by compressing or compacting a tablet core into the desired shape of the medicament. As used herein, “compact, compacting, or compacted” and “compress, compressing, or compressed” may be used interchangeably to describe the commonly used process of compacting powders into tablets via conventional pharmaceutical tableting technology as well known in the art. One typical such process employs a rotary tablet machine, often referred to as a “press” or “compression machine”, to compact the powders into tablets between upper and lower punches in a shaped die. This process produces a core having two opposed faces, formed by contact with an upper and lower punch, and having a belly band formed by contact with a die wall. Typically such compressed tablets will have at least one dimension of the major faces at least as long as the height of the belly band area between the major faces. Alternately, processes have been disclosed in the prior art to enable the “longitudinal compression” of tablet cores. When longitudinally compressed tablets are employed, it has been found that an aspect ratio (height between the major faces to width or diameter of the major faces) from about 1.5 to about 3.5, e.g., about 1.9 facilitates handling.

Other processes for producing the core may include confectionary processes such as those typically used for gums and lozenges, such as roping and cutting or molding.

Tablets are typically compacted to a target weight and “hardness”. Hardness is a term used in the art to describe the diametrical breaking strength as measured by conventional pharmaceutical hardness testing equipment, such as a Schleuniger Hardness Tester. In order to compare values across differently sized tablets, the breaking strength is normalized for the area of the break (which may be approximated as tablet diameter times thickness). This normalized value, expressed in kp/cm2, is sometimes referred in the art as “tablet tensile strength.” A general discussion of tablet hardness testing is found in Leiberman et al., Pharmaceutical Dosage Forms - Tablets, Volume 2, 2nd ed., Marcel Dekker Inc., 1990, pp. 213 - 217, 327 - 329, which is incorporated by reference herein.

The medicaments manufactured according to the present invention, therefore, provide the desired shape, swallowability and appearance for a solid dosage form. Further, the dosage form of the invention provides improved onset of dissolution and disintegration, while not compromising swallowability of the dosage form. Use of the dosage form in accordance with the invention permits the ability to add actives, colors, flavors, sensates and textures; impart improved swallowability, perception of speed, taste masking, and visual recognition to aid in product selection.

The process of the invention may produce a tablet that comprises a cavity or cavities and/or deposited portion(s)s on two sides or faces of the tablet, such as on the bottom and top of the tablet. The tablet may have the same amount or different amounts of cavities and/or deposited portions on the top and bottom of the tablet. In the process, the tablet may be handled such that the deposited portions stay in place or do not migrate between deposition on each face. This may be accomplished by a first deposition on one face; and the addition of a cooling, solidification or drying step and then a second deposition on the second face. This may also be accomplished by orienting or through a captive or positionally controlled rotation of the tablet so that portions can be deposited on the second face. In some examples a combination of (1) a first deposition on one face of the tablet; (2) cooling, solidification and/or drying of the first deposited portion(s), (3) positionally controlled rotation of the tablet, (4) a second deposition on a second face and (5) cooling, solidification and/or drying of the second deposited portion(s) are utilized. This deposition process can be repeated to build the deposited layers to increase the amount of active ingredients or combine different active ingredients within different layers.

The process of the invention may also produce a tablet that comprises cavitiy/cavities on one side or face of the tablet, such as on the top of the tablet, and an alignment feature and/or an identification feature on the opposite side of the tablet, such as on the bottom of the tablet.

The deposited portion may be added by a variety of methods. These methods include solution depositing, suspension depositing, melt depositing, ink jet printing, 2D printing or 3D printing. In the case of depositing, the flowable material will be metered out using a specialized pump and a nozzle or printing head. The deposition solution may be maintained in a reservoir that feeds either single or multiple channel pumps. The deposition solution is metered through a nozzle through either volumetric or gravimetric displacement within the pump. The combination of deposition pump displacement distance, speed, and nozzle size defines the volume of deposition solution deposited into the cavity. Variable deposition volumes can be accomplished in situ through changes to the pump displacement distance and speed. The pump displacement is a combination of forward and reverse positioning within a single deposition to mitigate potential impacts associated with droplet size and surface tension within the process. In this manner, the droplet volume, and associated deposition volume, can be controlled outside the constraints of deposition solution surface tension.

In instances wherein the flowable material is deposited on several sides of the dosage form, the dosage form or tablet must be positioned and oriented. Methods for positioning and inspection include vision monitoring systems and controls. Methods for orienting include but are not limited to captive carrier trays, pucks transported on a conveying belt or through the use of robotic transfer through a deposition zone.

The dosage form may be moved or oriented in between deposition steps to accommodate deposition into different cavities, or deposition of various compositions into separate cavities. The cavities may be positioned in a line along a surface of a dosage form; e.g. at least two cavities positioned longitudinally along the face of a tablet.

In instances wherein the flowable portion is deposited as a solution or suspension, water or solvent may require removal through the use of a drying step. Suitable drying steps may include infrared heat, convection drying, radio-frequency heating, or microwave heating.

It will become apparent to those skilled in the art that various modifications to the examples of the invention can be made by those skilled in the art without departing from the spirit or scope of the invention as defined by the appended claims.

### EXAMPLES

### Tablet Core: Formulation and Deposition

Part A: Placebo Core Tablets prepared by blending lactose and microcrystalline cellulose
Trials with placebo (Lactose & Microcrystalline Cellulose blend) with Opadry@ White 03U180000 film-coating.
Core is prepared at approximately 0.2425 inches x 0.0980 inches x 0.0600 inches half oval cavity.

PART A1: Core Tablet Containing an Active Ingredient(s)
Create a uniform blend of loperamide hydrochloride (2 mg) with 0.5-1% magnesium stearate, and lactose & microcrystalline cellulose as described in Part A; alternately create a uniform of blend of calcium carbonate and magnesium stearate (up to 99%) with additional typical other excipients.
Compress into core with the dimensions described in Part A.

### Deposition Parameters

### Part B: Deposition Step

The cores from Part A or A1 are deposited using the solutions in Example(s) 1-7. Deposition is completed with an IVEK 40 Pitch linear actuator and 3A pump with a DS3020 controller. The nozzle is a 20GA blunt needle. The core tablets are kept on an angled platform and dispensed on one side only using a custom script linked to the translation head of a Jetlab 4 printer from Microfab Technologies, Inc. The parameters used for the IVEK pump can be seen in Table 1.

TABLE 1

IVEK Pump Parameters

| | |
|---|---|
| Parameter | Setpoint |
| Direction | Forward |
| Dispense Volume | S1-µL / S2-8.2 µL |
| Dispense Meter rate | 50 µL/s |
| Load rate | 50 µL/s |
| Load Threshold | 50 µL |
| Drawback | Disabled |

### Example 1: Simethicone Encapsulation in Sugar Alcohol Via Melt Deposition

The following process can be followed to encapsulate simethicone on the tablet:
1. Dose of 10 mL simethicone into tablet cavity.
2. Load sugar alcohol at temperatures listed in Table 1 into Ivek pump (102118-2110).
3. Position Ivek nozzle tip between 1 - 2 mm from the simethicone surface.
4. Set Ivek deposition per stroke to 5 Ml.
5. Deposit sugar alcohol at a speed of 1000 rpm for 5.67 forward strokes followed by 4.67 reverse strokes.
6. Allow > 5 seconds for the sugar alcohol to crystallize/solidify.

### Example 2: Simethicone Encapsulation in Sugar Alcohol Via Melt Atomizing Deposition

The following process can be followed to encapsulate simethicone within a cavity:
1. Dose 10 mL of simethicone into tablet cavity.
2. Load sugar alcohol at temperatures listed in Table 1 into Ivek Sonicair nozzle (142658-28).
3. Position Ivek nozzle tip between 1 - 3 mm from the simethicone surface.
4. Deposit 0.2 mL of sugar alcohol on the simethicone surface while translating the nozzle or cavity.
5. Allow > 1 second for the sugar alcohol to crystallize/solidify.
6. Replicate process 14 times to deposit a total of 3 mL of sugar alcohol.

### Example 3: Simethicone Encapsulation in Sugar Alcohol Via Melt Jetting Deposition

The following process can be followed to encapsulate simethicone within a cavity:
1. Dose 10 mL of simethicone into tablet cavity.
2. Load sugar alcohol at temperatures listed in Table 2 into Vermes MDS 3280 micro dispensing system.
3. Position nozzle tip between 1 - 3 mm from the simethicone surface.
4. Deposit 1 mL of sugar alcohol on the simethicone surface while translating the nozzle or cavity.
5. Allow > 5 seconds for the sugar alcohol to crystallize/solidify.
6. Replicate process 2 times to deposit a total of 3 mL of sugar alcohol.

TABLE 2

Sugar alcohol formulations and associated deposition temperatures.

| Formulation | Material 1 | Material 1 wt. % | Material 2 | Material 2 wt. % | Deposition Temperature (°C) |
|---|---|---|---|---|---|
| A1 | Erythritol | 100 | - | 0 | 125 |
| B1 | Erythritol | 90 | Xylitol | 10 | 110 |
| C1 | d-Mannitol | 100 | - | 0 | 170 |
| D1 | d-Mannitol | 80 | Xylitol | 20 | 140 |
| E1 | d-Sorbitol | 100 | - | 0 | 100 |
| F1 | Xylitol | 100 | - | 0 | 95 |

### Example 4: Simethicone Encapsulation in Cellulose Derivative Via Atomizing Deposition

The following process can be followed to encapsulate simethicone within a cavity:
1. Dose 10 mL of simethicone into tablet cavity.
2. Prepare a 23 weight percent solution of Hypromellose succinate (HPMC-AS LG grade, commercially available from the Ashland Corporation) in acetone.
3. Load HPMC-AS solution into Ivek Sonicair nozzle (142658-28).
4. Position Ivek nozzle tip between 1 - 3 mm from the simethicone surface.
5. Deposit 0.2 mL of HPMC-AS solution on the simethicone surface while translating the nozzle or cavity.
6. Allow > 1 second for acetone evaporation.
7. Replicate process 5 times to deposit a total of 1 mL of HPMC-AS.

### Example 5: Simethicone Immobilization Via Network Swelling

The following process can be followed to immobilize simethicone within a cavity
1. Deposit 2 mL of the formulations listed in Table 3 into the tablet cavity.
2. Expose the cavity to < 420 nm light for 30 seconds in the presence of nitrogen.
3. Dose 10 mL of simethicone into the tablet cavity.
4. Allow > 600 seconds for simethicone to swell the networks formed from the formulations listed in Table 3.

TABLE 3

Photocurable network formulations (weight %)

| Formulation | m-PDMS-co-PCL | m-PDMS-co-PEG | DCM | DDFD |
|---|---|---|---|---|
| A2 | 50 | 0 | 46 | 4 |
| B2 | 40 | 10 | 46 | 4 |
| C2 | 0 | 50 | 46 | 4 |

m-PDMS-co-PCL: methacrylated-polydimethylsiloxane-co-polycaprolactone
m-PDSM-co-PEG: methacrylated-polydimethylsiloxane-co-polyethylene glycol
DCM: dichloromethane
DDFD: 4,4-dimethyldihydrofuran-2,3-diione

### Example 6: Glycerol Encapsulation in Cellulose Derivative Via Atomizing Deposition

The following process can be followed to encapsulate glycerol within a cavity:
1. Dose 10 mL of simethicone into tablet cavity.
2. Prepare a 15 weight percent solution of HPMC grade in water.
3. Load HPMC solution into Ivek Sonicair nozzle (142658-28).
4. Position Ivek nozzle tip between 1 - 3 mm from the simethicone surface.
5. Deposit 0.2 mL of HPMC solution on the simethicone surface while translating the nozzle or cavity.
6. Replicate process 5 times to deposit a total of 1 mL of HPMC.

### Example 7: Simethicone Encapsulation in Carrageenan Shell Via Ionic Crosslink Deposition

The following process can be followed to encapsulate simethicone on the tablet:
1. Prepare Carrageenan solution by combining 25.2 g of glycerin, 3.86 g of Carrageenan, and 1.65 g of Locust Bean Gum.
2. Prepare a second potassium sorbate solution by dissolving 2.1 g of potassium sorbate in 252 g of deionized water heated to 85° C.
3. Load carrageenan solution and potassium sorbate solution into separate reservoirs of a Vermes Microdispensing system (MDV 3283-FH).
4. Dose 10 mL of simethicone into tablet cavity.
5. Position both Vermes nozzle tips between 1 - 2 mm from the simethicone surface.
6. Dose both systems in concert to a total volume of 1 mL.
7. Allow > 10 seconds for the carrageenan mixture to solidify.

1. A dosage form comprising a substrate with at least one cavity, wherein the at least one cavity comprises simethicone and at least one material to immobilize the simethicone.

2. The dosage form of claim 1, wherein the substrate is a tablet core.

3. The dosage form of claim 1 wherein the dosage form comprises at least two cavities.

4. The dosage form of claim 1 wherein the dosage form comprises at least two cavities, and wherein at least one cavity is on an opposing surface.

5. The dosage form of claim 1, wherein the dosage form comprises four cavities on each of the opposing surfaces.

6. The dosage form of claim 3, wherein the at least two cavities are separated by at least a 1 mm portion of the surface of the substrate.

7. The dosage form of claim 1, wherein the substrate is elongated.

8. The dosage form of claim 1, wherein the dosage form comprises loperamide.

9. The dosage form of claim 1 ,wherein the dosage form comprises calcium carbonate, aluminum hydroxide, magnesium stearate, magnesium hydroxide or combinations thereof.

10. The dosage form of claim 3 , wherein the at least two cavities are elongated along the same axis as the elongated substrate.

11. The dosage form of claim 3, wherein the at least two cavities are equal in size.

12. The dosage form of claim 1 , wherein the substrate is coated.

13. The dosage form of claim 1 , wherein the material to immobilize the simethicone is a polymer, monomer, polyol, or gelling material.

14. A method of making a dosage form, comprising:
(a) preparing a substrate with two opposing surfaces and at least one cavity;
(b) depositing a flowable material comprising simethicone into at least one cavity on one side of the substrate; and
(c) adding a second material to immobilize the simethicone.

15. The method of claim 14, wherein the second material is deposited on the surface of the simethicone.

16. The method of claim 14, wherein the second material is photocurable.

17. The method of claim 14, wherein the second material is capable of ionic gelation.

18. The method of claim 14,wherein the second material melts at less than 120° C.

19. The method of claim 14, further comprising an additional drying step.

20. The method of claim 14, wherein the substrate comprises loperamide.

21. The method of claim 14, wherein loperamide is deposited into a second cavity.

22. The method of claim 14, wherein the substrate comprises calcium carbonate, aluminum hydroxide, magnesium stearate, magnesium hydroxide or combinations thereof.

23. A method of making a dosage form, comprising:
(a) preparing a substrate with two opposing surfaces and at least one cavity;
(b) depositing a flowable material comprising a mixture of simethicone and a second material into at least one cavity on one side of the substrate; and
(c) immobilizing the mixture of simethicone and second material through ionic gelation, photocuring, solution evaporation, drying or cooling.
